(19) United States
(12) Patent Application Publication
(10) Pub. No.: US 2006/0009835 A1
(43) Pub. Date: Jan. 12, 2006

(54) Graft, stent graft and method

(76) Inventors: Thomas A. Osborne, Bloomington, IN (US); Shyam S.V. Kuppurathanam, Bloomington, IN (US)

Correspondence Address: BRINKS HOFER GILSON & LIONE, P.O. BOX 10395, CHICAGO, IL, 60610, US

(21) Appl. No.: 11/176,570

(22) Filed: Jul. 7, 2005

Related U.S. Application Data
(60) Provisional application No. 60/586,036, filed on Jul. 7, 2004.

(51) Int. Cl. A61F 2/06; A61F 2/86

(52) U.S. Cl. 623/1.13; 623/1.54; 623/1.51

(57) ABSTRACT

The invention is directed to grafts, including graft fabrics, for implanting, transplanting, replacing or repairing a part of a patient. The present invention is also directed to stent grafts and endoluminal prostheses formed of the graft fabric. The problem of treating small and tortuous vessels of a patient with stent grafts is solved by providing a graft fabric comprising a plurality of yarns, the yarns having a low denier, wherein the graft and endoluminal prostheses comprising graft fabric can be packable in a delivery system having an outer diameter of from about 0.06 inches to 0.27 inches.

### RELATED APPLICATIONS

The present patent document claims the benefit of the filing date under 35 U.S.C. §119(e) of Provisional U.S. Patent Application Ser. No. 60/586,036, filed Jul. 7, 2004, which is hereby incorporated by reference.

### BACKGROUND

1. Technical Field

This invention is directed to grafts for implanting, transplanting, replacing, or repairing a part of a patient and to stent grafts and endoluminal prostheses formed of the graft. The present invention is also directed to methods of making the grafts and the stent grafts.

2. Background Information

Identification of materials suitable for grafts can be difficult, because such materials must possess disparate properties. For example, vascular graft materials should exhibit mechanical stability under continuous stress, should have compliance similar to that of the host tissue, and should be non-thrombogenic. In some applications, graft materials may also provide for endothelialization and have sufficient porosity to allow for capillarization. In other applications, graft materials may provide for a reduced permeability to blood. Other preferred properties of graft materials include being non-allergenic and non-carcinogenic. While all of these properties may be specifically designed into a graft material, it is also desirable for the material to be inexpensive to fabricate.

Portions of the human vasculature may be replaced or treated with synthetic vascular grafts. Synthetic vascular grafts may have a wide variety of configurations and may comprise a wide variety of materials. Conventional vascular graft implants include those formed from a biologically compatible material and which retain an open lumen to permit blood to flow there through after implantation. Polymeric structures typically used for vascular graft materials and stent procedures may include woven and non-woven textile materials.

The delivery of vascular implants to a specific location in the vascular tree of a patient is essentially limited by the diameter, tortuosity and access to the vessel to be treated. Vascular implants for delivery into relatively large vessels (6 millimeter (mm) diameter or larger) are well-known in the art. For example, polyethylene terephthalate fiber fabric arrays have been utilized for such application. Polyethylene terephthalate fiber fabrics are manufactured, for example, by the DuPont Company (often known as “DACRON™” when manufactured by DuPont). Similar fabrics are also manufactured by other companies from various substances. Examples of commercially available woven and knitted fabrics of medical grade Dacron fibers include, for example, single and double velour graft fabrics, stretch Dacron graft fabric and Dacron mesh fabrics.

However, there is still a need for vascular grafts for implantation into small and tortuous vessels (less than 6 mm diameter). Such implantation necessitate that the implant be capable of being collapsed into a sufficiently small diameter delivery system and being expanded at a desired location in a patient while retaining all the desired properties for its intended purpose.

### SUMMARY

In one embodiment, the present invention is a graft comprising a graft fabric comprising a plurality of yarns, the yarns having a denier of 5 to 50, said graft being packable in an endovascular delivery system having an outer diameter of from about 0.06 inches (5 French) to about 0.27 inches (20 French); preferably from about 0.10 inches (8 French) to about 0.22 inches (17 French); and most preferably from about 0.13 inches (10 French) to about 0.19 inches (14 French). The yarns of the graft fabric may have a denier of 5 to 40 or 20 to 40. Preferably, the yarns comprise a synthetic polymer, such as a thermoplastic material. The thermoplastic material may comprise at least one of polyester, polypropylene, polyurethane and polytetrafluoroethylene. Preferably, the yarns are made from filaments or fibers having a low denier. Preferably, the filaments or fibers have a denier less than or equal to about 1.4. More preferably, the filaments or fibers have a denier of less than or equal to about 0.7. Most preferably, the filaments or fibers have a denier of less than or equal to about 0.4. Preferably, the yarns comprise a monofilament. The yarns may comprise multifilaments. The yarns may be textured or non-textured. Preferably, the yarns have a tenacity of about 4 grams per denier or more, more preferably of about 6 grams per denier or more. Preferably, the graft fabric of the graft may further comprise a hydrophilic material. The hydrophilic material may be mechanically bonded to a surface of the graft fabric. Alternatively, the hydrophilic material may be covalently bonded to the surface of the graft fabric. The yarns of the graft fabric may be woven or nonwoven. The weave of the graft fabric may be a plain weave, a matt weave, or a combination thereof. The weave type of the graft fabric may be uniform or non-uniform. The number of ends per inch may be less than about 152 and the number of picks per inch may be less than about 135.

In another embodiment, the invention is a graft comprising a graft fabric comprising a plurality of yarns, the yarns having a denier of 5 to 50, the yarns being made from filaments or fibers having a denier less than or equal to about 1.4 and comprising a synthetic polymer. The graft fabric further comprises a hydrophilic material. Preferably, such graft is packable in an endovascular delivery system having an outer diameter of from about 0.06 inches to about 0.27 inches.

In another embodiment, the invention is an endoluminal prosthesis, comprising a tubular graft comprising a graft fabric comprising a plurality of yarns, the yarns having a denier of 5 to 50; and a stent supporting the graft fabric. The endoluminal prosthesis is packable in a delivery system having an outer diameter of from about 0.06 inches (5 French) to about 0.27 inches (20 French), preferably from about 0.10 inches (8 French) to about 0.22 inches (17 French), and most preferably from about 0.13 inches (10 French) to about 0.19 inches (14 French). The yarns of the graft fabric may have a denier of 5 to 40 or 20 to 40. Preferably, the yarns of the graft fabric of the endoluminal prosthesis comprise a synthetic polymer. The synthetic polymer may be a thermoplastic material comprising at least one of polyester, polypropylene, polyurethane and polytetrafluoroethylene. Preferably, the graft fabric is polyester. The yarns of the graft fabric may be made of filaments or fibers having a low denier. Preferably, the filaments or fibers have a denier of less than or equal to about 1.4. More preferably, the filaments or fibers have a denier of less than or equal to about 0.7. Most preferably, the filaments or fibers have a denier of less than or equal to about 0.4. Preferably, the yarns of the graft fabric comprise a monofilament. The yarns of the graft fabric may also comprise multifilaments. The yarns may be textured or non-textured. Preferably, the yarns of the graft fabric of the endoluminal prosthesis have a tenacity of about 4 grams per denier or more, or about 6 grams per denier or more. Preferably, the graft fabric of the endoluminal prosthesis may further comprise a hydrophilic material. The hydrophilic material may be mechanically bonded to the graft fabric. The hydrophilic material may be covalently bonded to the graft fabric of the endoluminal prosthesis. Hydrophilic material may act as a lubricant for facilitating introduction of the prosthesis inside an endovascular delivery system and delivery of the prosthesis to a precise location within the body of a patient. Also, activation of the hydrophilic material causes swelling of the material and provides, as a result, means to reduce the porosity and the permeability of the graft fabric. Although the hydrophilic material may be often activated before implantation, body fluids in contact with the graft fabric after implantation often tend to maintain the swelling of the hydrophilic material. The yarns of the graft fabric of the endoluminal prosthesis may be woven or nonwoven. The weave may be a plain weave, a matt weave, or a combination thereof. The weave type of the yarns of the tubular graft fabric of the endoluminal prosthesis may be uniform or non-uniform. Preferably, the number of ends per inch is less than about 152 and the number of picks per inch is less than about 135. Preferably, the tubular graft fabric of the endoluminal prosthesis has, after implantation of the prosthesis into a vascular lumen of a patient, a permeability of about zero mL/min/cm2 to about 240 mL/min/cm2. More preferably, the tubular graft fabric of the endoluminal prosthesis has, after implantation of the prosthesis into a vascular lumen of a patient, a permeability of about 80 mL/min/cm2 to about 240 mL/min/cm2. Most preferably, the tubular graft fabric of the endoluminal prosthesis has, after implantation of the prosthesis into a vascular lumen of a patient, a permeability of about 160 mL/min/cm2 to about 240 mL/min/cm2.

In yet another embodiment, the invention is an endoluminal prosthesis comprising a tubular graft, which comprises a graft fabric and a stent supporting the graft fabric. The graft fabric comprises a plurality of yarns, the yarns having a denier of 5 to 50, the yarns being made from filaments or fibers having a denier less than or equal to about 1.4, and comprising a synthetic polymer. The graft fabric further comprises a hydrophilic material. The endoluminal prosthesis is packable in an endovascular delivery system having an outer diameter of from about 0.06 inches to about 0.27 inches.

In yet another embodiment, the present invention is a method for making an endoluminal graft prosthesis for implantation into a patient. The method comprises the steps of providing a graft comprising a graft fabric having a plurality of yarns, the yarns having a denier of form 5 to 50; treating the graft fabric with a hydrophilic material; attaching a stent to the graft fabric to form a stent graft; and inserting the stent graft into an endovascular delivery system. The diameter of the endovascular delivery system is preferably from about 0.06 inches (5 French) to about 0.27 inches (20 French), more preferably from about 0.10 inches (8 French) to about 0.22 inches (17 French), and most preferably from about 0.13 inches (10 French) to about 0.19 inches (14 French). The yarns of the graft fabric may have a denier of 5 to 40 or 20 to 40. Preferably, the yarns are made from filaments or fibers having a denier less than or equal to about 1.4. More preferably, the yarns are made from filaments or fibers having a denier less than or equal to about 0.7. Most preferably, the yarns are made from filaments or fibers having a denier less than or equal to about 0.4. The yarns may comprise a monofilament or multifilaments. The yarns may be textured or non-textured. The filaments or fibers of the yarns of the graft fabric may comprise a synthetic polymer, such as a thermoplastic material. The thermoplastic material preferably comprises at least one material selected from the group consisting of polyester, polypropylene, polyurethane, and polytetrafluoroethylene. Preferably, the yarns of the graft fabric have a tenacity of about 4 grams per denier or more; more preferably, about 6 grams per denier or more. The step of treating the graft fabric with the hydrophilic material may comprise positing the hydrophilic material on at least one surface of the graft fabric. Preferably, the step of treating the graft fabric with the hydrophilic material may include mechanically bonding the hydrophilic material to the surface of the graft fabric. Alternatively, the step of treating the graft fabric with the hydrophilic material includes covalently bonding the hydrophilic material to the surface of the graft fabric. The yarns of the graft fabric may be woven or nonwoven. The weave may be one of a plain weave, a matt weave, or a combination thereof. Preferably, the weave type is uniform. Alternatively, the weave type may be non uniform. The number of ends per inch may be less than about 152 and the number of picks per inch may be less than about 135. Preferably, the step of attaching the graft fabric to a stent further includes attaching the stent prior to the step of treating the graft fabric with the hydrophilic material. Alternatively, the step of attaching the graft fabric to a stent further includes attaching the stent after the step of treating the graft fabric with the hydrophilic material.

### BRIEF DESCRIPTION OF THE DRAWINGS

FIG. 1 is an illustration of exemplary types of weave.

FIG. 2 is a schematic illustration of a fragmentary, perspective view of a single layer of woven fabric showing an exemplary distribution of yarns.

FIG. 3 shows a modular bifurcated aortic endoluminal prosthesis comprising the graft fabric of the present invention, implanted within an aneurysm of the aorta.

FIG. 4 shows a fabric stent graft covered by a layer of hydrophilic material.

FIG. 5 shows a non-bifurcated aortic endoluminal prosthesis comprising the graft fabric of the present invention.

### DETAILED DESCRIPTION OF THE DRAWINGS AND THE PRESENTLY PREFERRED EMBODIMENTS

Unless defined otherwise, all technical and scientific terms used herein have the same meaning as commonly understood to one of ordinary skill in the art to which this invention belongs.

The term “graft” means any replacement for a bodily tissue or for a function of a bodily tissue. A graft may be transplanted from a donor to a recipient to repair a part of a body, and in some cases, the patient can be both donor and recipient. For example, a graft may replace tissue that has been destroyed or create new tissue where none exists.

The term “yarn” refers to a length of a continuous thread or strand of one or more filaments or fibers, with or without twist, suitable for weaving, knitting or otherwise intertwining to form a textile fabric. The term “textured yarn” refers to a yarn that has been processed to create durable fine distortions along the length of the yarn, such as creases, crimps, coils, loops, spirals, twists or other. In the present description, the term yarn encompasses yarns, filaments, fibers, threads, strands, and the like.

The term “fabric” refers to a type of graft material. The fabric may be a textile. The fabric may be woven or nonwoven. The woven fabric may be produced by weaving or knitting filaments. “Weaving” refers to forming a fabric by interlacing filaments in one direction (warp) with others at a right angle to them (weft, fill or filling). A “weave” refers to the pattern of interlacing warp and weft in a woven fabric, which may be for example a plain weave (one up, one down), a matt weave (two up, two down) or a combination thereof. A weave may be uniform if the number of ends (i.e. number of yarns, filaments or fibers in the direction of the warp) per inch is equal to the number of picks (i.e. number of yarns, filaments or fibers in the direction of the weft, fill or filling) per inch.

The term “denier” refers to the mass in grams of 9000 meters of a yarn.

The term “tenacity” or “tensile strength” refers to the ability of a yarn to resist breaking under tension. The tenacity of a fabric may be measured in the direction of the warp or in the direction of the weft.

The “permeability” of a fabric refers to the amount of water (saline) measured in milliliters that passes through a square centimeter of the fabric in 60 seconds (ml/min/cm2), under physiological pressure (typically 120 mm Hg).

The terms “patient”, “subject,” or “recipient” as used in this application refers to animals, particularly to mammals, and especially to humans.

The present invention is directed to grafts, comprising graft fabric, and endoluminal prostheses that include the graft fabric, for implanting into small and tortuous vessels. Implantation into small and tortuous vessels necessitate that the grafts and endoluminal prostheses be capable of being collapsed into a sufficiently small diameter endovascular delivery system and capable of being expanded at a desired location in a patient while retaining all the desired properties for its intended purpose. Accordingly, the graft fabric comprises plurality of modified yarns having a low denier of 5 to 50. Also the filaments or fibers that make up the graft fabric may have a low denier. A reduced number of filaments per yarn may also be appropriate for such application.

For small vascular graft applications (less than 6 mm diameter), and for other applications for which suitable substrates of desired structure are not commercially available, special manufacture of graft fabrics having suitably small diameter and other properties in accordance with the present invention may be necessary.

Fabric Structures

A graft of this invention may include a graft fabric of biocompatible material. The term “biocompatible” refers to material that is substantially non-toxic in the in vivo environment of its intended use, and that is not substantially rejected by the patient's physiological or immunological system (i.e. is not antigenic). This can be gauged by the ability of a material to pass biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible material, when introduced into a majority of patients, will not cause a significantly adverse, long-lived or escalating biological reaction or response, and is distinguished from a mild, transient inflammation which typically accompanies surgery or implantation of foreign objects into a living organism.

Examples of biocompatible textile fabric materials from which graft fabric of the present invention can be formed include polyesters, such as polypropylene and polyethylene terephthalate; fluorinated polymers, such as polytetrafluoroethylene (PTFE) and fibers of expanded PTFE; and polyurethanes. In addition, materials that are not inherently biocompatible may be subjected to surface treatments or modifications in order to render the materials biocompatible. Thus, any fibrous material may be used to form a graft fabric, provided the final graft fabric is biocompatible.

Polymeric materials that can be formed into fibers suitable for making yarns and fabrics include, for example, polyethylene, polypropylene, polyaramids, polyacrylonitrile, nylons and cellulose, in addition to polyesters, fluorinated polymers, polyurethanes as listed above, and other suitable polymeric materials. Preferably, the fabric is made of one or more polymers that do not require treatment or modification to be biocompatible. More preferably, the fabric is made of biocompatible polyester. Most preferred fabrics include those formed from polyethylene terephthalate and PTFE. These materials are inexpensive, easy to handle, have good physical characteristics and are suitable for clinical application.

As previously mentioned, the fabrics may be nonwoven or woven (including knitted) fabrics.

Nonwoven fabrics are fibrous webs that are held together through bonding of the individual yarns. The bonding can be accomplished through thermal or chemical treatments or through mechanically entangling the yarns. Because nonwoven fabrics are not subject to weaving or knitting, filaments or fibers can be used in a crude form without being converted into a yarn structure. Woven fabrics are fibrous webs that have been formed by knitting or weaving.

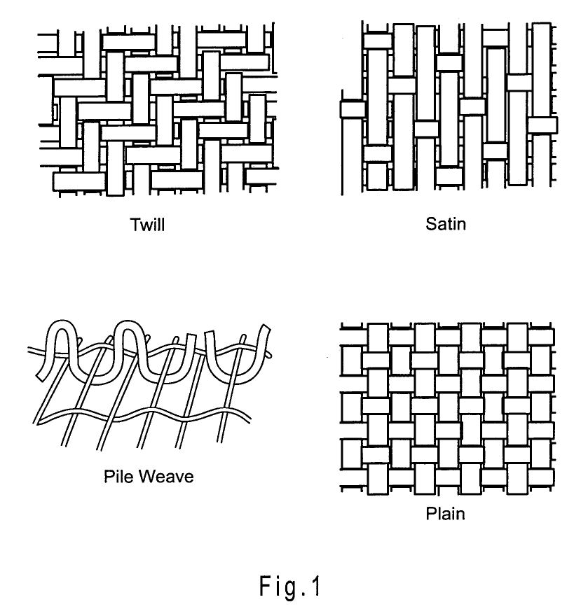

The woven fabric structure may be any kind of weave including, for example, a plain weave, a matt weave, a herringbone weave, a satin weave, a pile, a twill, or a basket weave. FIG. 1 illustrates plain, satin, pile, and twill weaves. The preferred weave for the graft fabric of the present invention include plain weave, matt weave, or a combination thereof.

The weave type may be uniform, but is preferably non-uniform with a number of yarns in the direction of the warp (ends per inch) that is less than about 152 and a number of yarns in the direction of the weft (picks per inch) that is less than about 135.

The choice of a weave may depend on the physical properties desired for a graft. For example, the choice of weave may depend on the type of weave, the diameter of the spaces between yarns, the shape of the yarns, the finishing techniques, the presence or absence of a coating, such as a hydrophilic coating, and the configuration of the graft fabric (e.g. a single layer or multi-layer graft fabric and relative direction of the yarns, fibers or filaments in the multi-layered graft fabric). For example, if it is desired to obtain a graft fabric that has low permeability, preferably of less than 240 mL/min/cm2, then one would choose, for example, a plain weave, 40 denier warp and fill yarn, 100-150 ends per inch, 100-150 picks per inch, and a filament yarn type.

In one example of woven fabrics, knitted fabrics include weft knit and warp knit fiber arrays. Weft knit fabric structures (including double-knit structures) utilize interlocked fiber loops in a filling-wise, or weft, direction, while warp knit structures utilize fabric loops interlocked in a length wise, or warp, direction. Weft knit structures generally are more elastic than warp knit structures, but the resiliency of warp knit fabrics is satisfactory to provide a substantial degree of elasticity, or resiliency, to the fabric structure without substantially relying on tensile fiber elongation for such elasticity. Weft knit fabrics generally have two dimensional elasticity (or stretch), while warp knit fabrics generally have unidirectional (width wise) elasticity. The different elasticity properties of the various knit or woven structures may be beneficially adapted to the functional requirement of the particular graft fabric application. In some cases, where little elasticity is desired, the fabric may be woven to minimize in plane elasticity but yet provide flexibility.

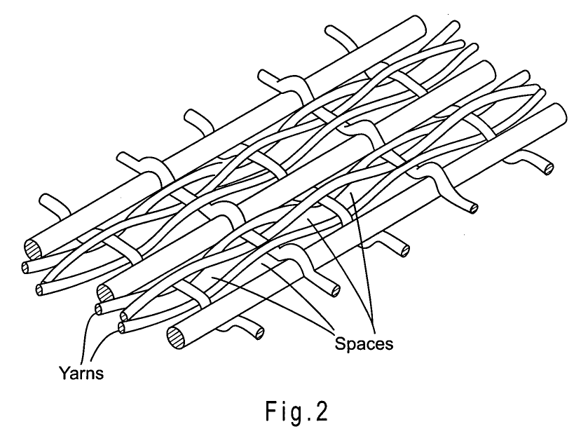

As illustrated in FIG. 2, preferably the graft fabric of this invention may be a fabric that includes yarns and spaces between the yarns.

The yarns are made up of filaments or fibers that are either woven or nonwoven, as described above. Preferably, the filaments or fibers have a low denier. For example, the filaments of fibers making up the yarns may have a size of less than or equal to about 1.4 denier. More preferably, the size of the filaments or fibers may be less than or equal to about 0.7 denier. Most preferably, it may be less than or equal to about 0.4 denier. Preferably, the yarns have a denier of about 5 to about 50. More preferably, yarns have a denier of about 5 to about 40, and most preferably, the yarns have a denier of about 20 to about 40. Surprisingly, the low denier of filaments and yarns allows for formation of fabric materials that are thin, yet the materials can retain all the desired properties necessary for its use as graft fabric to make the graft and endoluminal prostheses of this invention. Preferred denier of the yarns provides for a thin structure that may be easily packable into an endovascular delivery system constructed for delivery of the graft and endoluminal prosthesis into small and torturous vessels. For example, the fabrics can be used to form a graft and endoluminal prosthesis packable in an endovascular delivery system having an outer diameter of from about 0.06 inches (5 French) to about 0.27 inches (20 French); more preferably an endovascular delivery system having an outer diameter of from about 0.10 inches (8 French) to about 0.22 inches (17 French); and most preferably, an endovascular delivery system having an outer diameter of from about 0.13 inches (10 French) to about 0.19 inches (14 French).

In graft fabrics, the filaments provide a flexible array in sheet or tubular form so that the graft fabric is provided with a predetermined high degree of flexibility. Furthermore, a high degree of elasticity may be provided through bending of the filaments of the array rather than through substantial tensile elongation of the filaments.

The yarns may comprise a monofilament or multifilaments.

The yarns may be textured or non-textured.

Preferably, the spaces between the yarns of the fabric comprise an average diameter from about 1 micron to about 400 microns. More preferably, the spaces between the yarns of the fabric comprise an average diameter from about 1 micron to about 100 microns. Most preferably, the spaces between the yarns of the fabric comprise an average diameter from about 1 micron to about 10 microns.

Because of the presence of spaces between the yarns of the fabric graft, graft fabrics have a porosity that is related to the type of yarn(s) and the physical characteristics of the yarns (diameter, shape, denier, etc.), the weave pattern and the finishing techniques. Generally speaking, the permeability to water of a fabric material is correlated to the porosity of the material. For example, known graft fabric materials, made of woven polyester and having a twill weave, have a permeability of about 350 ml/min/cm2 (available from VASCUTEK@ Ltd., Renfrewshire, Scotland, UK).

Woven fabrics for use as graft fabrics to form grafts of this invention may have any desirable size, shape, form, and configuration. For example, the filaments forming yarns of woven fabrics may be filled or unfilled. Examples of how the basic unfilled filaments may be manufactured and purchased are indicated in U.S. Pat. No. 3,772,137 by Tolliver. Certain physical parameters may be used to further characterize fabric filaments or fibers used in grafts of this invention. For example, fibers may be characterized as having a tensile strength (i.e., tenacity) and tensile modulus. Preferably, the fibers making up the yarns of the graft fabric have a tensile strength of at least about 20,000 psi and a tensile modulus of at least about 2×106 psi. Preferably, the yarns have a tenacity of about 4 grams per denier or more. Most preferably, the yarns have a tenacity of about 6 grams per denier or more. Preferably, the fabric is made of medical grade synthetic polymeric materials described above. The filaments or fibers of the fabric may also have a high degree of axial orientation. Fibers generally used in graft fabrics for medical use may be of a diameter from about 1 micron to about 5 millimeters.

Graft fabrics, in accordance with the present invention, preferably include a plurality of yarns. The yarns of the graft fabric may all be formed from the same material (e.g. polyester) or may be formed from yarns of different materials. In the latter case, multiple embodiments are encompassed by the present invention. In one embodiment of the present invention, only the yarns in the direction of the warp are made of different materials, whereas the yarns in the direction of the weft are all of the same material. In another embodiment, only the yarns in the direction of the weft are made of different materials, whereas the yarns in the direction of the warp are all of the same material. In yet another embodiment, the yarns of the warp and the weft are made of different materials.

Determination of which combination of materials in which direction of the fabric is most appropriate is based on the type of clinical application envisaged by the person skilled in the art, properties of the graft material that are desired, and further factors such as the weave type, the diameter or denier of the yarns, fibers or filaments, the shape of the yarns, fibers or filaments, the finishing techniques and/or permeability of the textile. For example, for percutaneous application, thin fabrics of this invention are desired. Such thin fabrics comprise yarns that have a low number of filaments and/or filaments that have a low denier as specified above.

Surprisingly, grafts and endoluminal prostheses that comprise thin graft fabrics are easily collapsable into a small diameter endovascular delivery system and are expandable at a desired location in a patient while retaining all the desired properties for their intended purpose.

Next, for certain clinical applications, it may be preferable to use graft fabrics having low porosity in order to achieve low permeability of the graft. Low porosity may be an inherent property of the graft fabric itself. Alternatively, low porosity may be obtained by treating the porous graft fabric, for example by coating with a material that fills the spaces. Such treatment further decreases the inherent porosity of the graft fabric to a desired level.

For example, the graft fabric of the present invention may be coated with a hydrophilic material that further reduces the porosity of the fabric, especially after activation and swelling of the hydrophilic material. The graft fabric of the present invention has preferably a permeability of less than 240 ml/min/cm2, which can be decreased to substantially zero ml/min/cm2 when a suitable hydrophilic material is used in appropriate quantity and layers.

Alternatively, to reduce the porosity of the graft fabric material, more yarns per cross section may be provided. To do so, the type of weave, ends per inch, picks per inch, and yarn cross section may be modified.

Coatings

In one embodiment of the present invention, as mentioned above, the graft fabric may further include a coating posited over the graft material. The coating may include, for example, a biocompatible hydrophilic material, such as hydrophilic polymer. Hydrophilic polymers that may be suitable for use as a coating for the graft fabric material of the present invention are readily and commercially available from, for example, Biosearch Medical Products, Sommerville, N.J.; Hydromer Inc. Branchburg, N.J.; Surmodics, Eden Prairie, Wis.; and STS Biopolymers, Inc., Henrietta, N.Y. For example, hydrophilic polymer may include, but not be limited to, polyethylene oxide, polyvinyl pyrrolidone, polyethylene glycol, carboxylmethyl cellulose, hydroxymethyl cellulose, and other suitable hydrophilic polymers, or a combination thereof.

Alternatively, the graft fabric may be impregnated or otherwise in contact with the hydrophilic material.

The hydrophilic material can act as a lubricant to facilitate introduction of the graft, including the graft fabric of the present invention into the endovascular delivery system. The hydrophilic coating may also facilitate extrusion of the graft or endoluminal prosthesis from the endovascular delivery system at an implantation site into a vessel of a patient. Further, the mere physical presence of the coating of hydrophilic material may serve to provide a barrier to the passage of fluids (e.g. body fluids) through the graft fabric. The coating material may also swell once in contact with blood or body fluids. After swelling of the material has occurred, the spaces between the yarns of the fabric may become occluded partially or totally. This in turn results in a further reduction of the permeability of the graft fabric. Therefore, it is possible to reduce the permeability of the fabric to a desired level and even to substantially zero ml/min/cm², by judiciously choosing the hydrophilic coating material, the graft fabric with a small average diameter of spaces and yarns, the shape of the yarns, the weave type, and the finishing techniques,

For example, porous fabric material may be coated and/or impregnated with a hydrophilic material to provide the final graft product. The term “impregnation” means providing for the presence of one or more components inside the porous fabric, in particular in the spaces of the fabric material. The coating and/or impregnation may be provided to reduce the porosity of the fabric at the time of implantation by acting as a filler to occlude the spaces between yarns of the graft fabric. By reducing the diameter of the spaces of a graft fabric, the hydrophilic material causes the graft to be less porous. This reduction in porosity results in lower permeability of the graft fabric to body fluids and blood, therefore minimizing blood loss through the graft.

The biocompatible hydrophilic coating layer may be posited over an entire surface or part of the surface of the graft fabric. The type of bonding between the graft fabric and the hydrophilic coating is preferably mechanical, although covalent bonding is also envisaged. Bonding of the hydrophilic material to the graft fabric is such that the hydrophilic material will remain on the graft at least until cells in the immediate vicinity of the graft have colonized the graft after implantation of the graft into a patient. After colonization of the graft by cells, the role of the hydrophilic material becomes secondary. The secondary role may be, for example, to lower permeability.

Methods of coating the graft fabric with a hydrophilic material are described below.

Methods of testing permeability of a graft material to water are well-known in the art. The results of such tests provide an indication of the ability of a graft to prevent passage of blood or other body fluids through the graft fabric of the implanted graft.

It should be understood that, in addition to or instead of the hydrophilic coating, the graft fabric may be coated, impregnated, or lined with, for example, bioactive agents to achieve desired physiological effects. In one embodiment, bioactive agents may be incorporated into or mixed with the hydrophilic material in a coating mixture for coating of the graft fabric. The bioactive agent may be present in a liquid, a finely divided solid, or any other appropriate physical form. In another embodiment, bioactive agents may be posited over or under the hydrophilic material. Optionally, the coating mixture may include one or more additives, for example, auxiliary substances, such as diluents, carriers, excipients, stabilizers, or the like.

Examples of bioactive agents include, without limitation, at least one of paclitaxel; estrogen or estrogen derivatives; heparin or another thrombin inhibitor; hirudin, hirulog, argatroban, D-phenylalanyl-L-poly-L-arginyl chloromethyl ketone or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; an antimicrobial agent or antibiotic; aspirin, ticlopidine or another anti-platelet agent; colchicine or another antimitotic, or another microtubule inhibitor; cytochalasin or another actin inhibitor; a remodeling inhibitor; deoxyribonucleic acid, an antisense nucleotide or another agent for molecular genetic intervention; GP IIb/IIIa, GP Ib-IX or another inhibitor or surface glycoprotein receptor; methotrexate or another antimetabolite or antiproliferative agent; an anti-cancer chemotherapeutic agent; dexamethasone, dexamethasone sodium phosphate, dexamethasone acetate or another dexamethasone derivative, or another anti-inflammatory steroid; dopamine, bromocriptine mesylate, pergolide mesylate or another dopamine agonist; 60Co (having a half life of 5.3 years), 192Ir (having a half life of 73.8 days), 32P (having a half life of 14.3 days), 111In (having a half life of 68 hours), 90Y (having a half life of 64 hours), 99 mTc (having a half life of 6 hours) or another radiotherapeutic agent; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha-tocopherol, superoxide dismutase, deferoxamine, a 21-aminosteroid (lasaroid) or another free radical scavenger, iron chelator or antioxidant; angiopeptin; a 14C-, 3H-, 131I-, 32P- or 36S-radiolabelled form or other radiolabelled form of any of the foregoing; an extracellular matrix, such as SIS (small intestine submucosa); or a mixture of any of these.

The bioactive agents may be released over time from the coating.

The amount of bioactive agent will be dependent upon a particular bioactive employed and medical condition to be treated. Typically, the amount of the bioactive agent represents about 0.001% to about 70% of the total coating weight, more typically about 0.001% to about 60% of the total coating weight. It is possible that the bioactive agent may represent as little as 0.0001% of the total coating weight.

Various methods of coating, impregnating, or lining the graft fabric with the bioactive agents may be utilized and are known in the art. For example, the bioactive agents may be deposited onto the graft fabric by spraying, dipping, pouring, pumping, brushing, wiping, vacuum deposition, vapor deposition, plasma deposition, electrostatic deposition, epitaxial growth, or any other method known to those skilled in the art. Methods for dip coating a medical device are disclosed, for example, in U.S. Pat. No. 6,153,252, which is incorporated by reference. The type of coating or vehicle utilized to immobilize the bioactive agent to the graft fabric may vary depending on a number of factors, including the type of the medical device, including the graft fabric, the type of bioactive agent, and the rate of release thereof.

In order to be effective, the bioactive agent should preferably remain on the graft fabric during the delivery and implantation of the prosthesis. Accordingly, various materials may be utilized as surface modifications to prevent the bioactive agent from coming off prematurely. These materials are known and commonly used in the art.

Endoprostheses

In one embodiment, the graft, including a graft fabric, of the present invention may be used to manufacture various medical devices, such as endoluminal graft prostheses. The endoluminal graft prosthesis may include endovascular grafts, stents, and combination stent-grafts. Preferably, endovascular grafts include those which comprise knitted or woven fabrics. The latter may be coated or impregnated with a variety of substances, including single or multiple bioactive or non-bioactive substances, as described above.

In a preferred embodiment, the endoluminal prosthesis of this invention includes a tubular graft comprising a graft fabric and a stent supporting the graft fabric. The graft fabric further comprises a plurality of yarns, the yarns having a denier of from 5 to 50, 4 to 40, or 20 to 40. Such endoluminal prosthesis may be packable in a delivery system having an outer diameter of from about 0.06 inches to about 0.27 inches; more preferably an outer diameter of from about 0.10 inches to about 0.22 inches; and most preferably an outer diameter of from about 0.13 inches to about 0.19 inches.

The stents of the prosthesis can be of the same type (i.e. self-expandable or balloon-expandable), or at least one stent of a multi-stent device can be of a different type than the remainder of the stents. At least one of the supporting stents can be a “hybrid” stent, which combines balloon expandable portion(s) and self-expandable portion(s). The graft fabric may be used to cover either the entire internal and/or external surface(s) of one or plurality of stents, or at least portion of the surfaces.

Although multi-layered fabrics can be used in various types of endoluminal prostheses, the devices of the present invention preferably comprise a single layer of graft fabric material. More preferably, the single layer of graft fabric material is seamless. However, if seams are present in fabrics making up the graft fabric of the present invention, the graft fabric may be coated with a hydrophilic material as described above. Hydrophilic materials are capable of swelling and filling the weave of the fabric after its activation upon implantation into a vessel, creating a seal that will substantially minimize and suppress leakage(s) at the seam(s).

The functional vessels of human and animal bodies, such as blood vessels and ducts, occasionally weaken or even rupture. For example, in the aortic artery, the vascular wall can weaken or tear, resulting in dangerous conditions such as aneurysms and dissections. Upon further exposure to hemodynamic forces, such an aneurysm can rupture. Treatment of such conditions can be performed by implanting a prosthesis within the vascular system using minimally invasive surgical procedures.

Often, bifurcated endoluminal prostheses are used for the treatment of vascular conditions near a branch point because a single, straight section of a tubular prosthesis may not be able to span the aneurysm or dissection and still maintain sufficient contact with healthy vascular tissue to secure the prosthesis and to prevent endoleaks. For example, most abdominal aortic aneurysms occur at or near the iliac bifurcation, and treatment with an endoluminal prosthesis requires the presence of prosthesis material in the main aorta and in the iliac branch arteries (Dietrich, E. B. J. Invasive Cardiol. 13(5):383-390, 2001). Typically, an endoluminal prosthesis for use near a bifurcation will have a main lumen body, for placement within the aorta, and two branch lumens extending from the main lumen body into the branch arteries. Similarly, bifurcated endoluminal prostheses may be used at or near branch point of small vessels.

One example of a bifurcated prosthesis in accordance with this invention is a single piece prosthesis. Such a unitary structure has a main tubular body and preformed leg extensions. The seamless structure provided by this configuration can minimize the probability of leakage within the prosthesis.

Another example of a bifurcated prosthesis is a modular system. In this system, one or both of the leg extensions can be attached to a main tubular body to provide the final prosthesis. Examples of modular systems are described in PCT Patent Application publication WO98153761 and in U.S. Patent Application publication 2002/0198587 A 1, which are incorporated herein by reference.

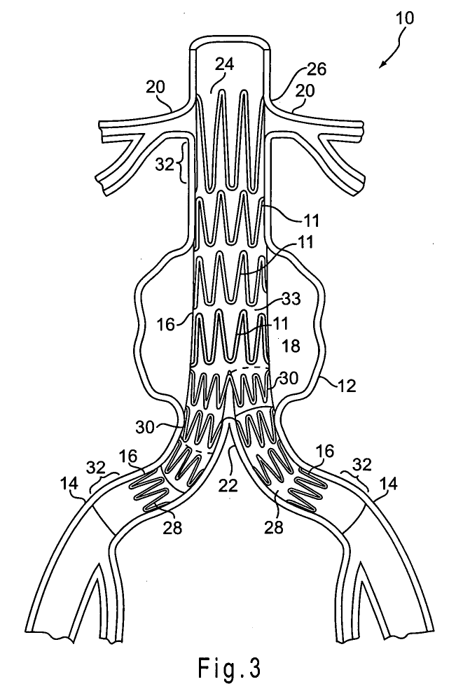

FIG. 3 shows an example of a modular bifurcated stent graft 10 deployed within an aneurysm of the aorta 12 and both iliac arteries 14. The prosthetic modules 16 that make up the stent graft 10 are generally tubular, so that the fluid can flow through the stent graft 10, and are preferably made of a textile 33, such as polyester, poly(ethylene terephthalate), or similar materials. The main body 18 extends from the renal arteries 20 to near the bifurcation 22. Multiple Z-stents 11 are sutured along the length of the stent graft 10. A suprarenal fixation stent 24 anchors the main body 18 to the healthier, preferably non-aneurysmal tissue 26 near the renal arteries. Two iliac extension modules 28 extend from the iliac limbs 30.

The stent graft 10 will preferably achieve a blood-tight seal at the contact regions 32 on both ends of the aneurysm 12, so that the aneurysm 12 will be excluded. In the particular embodiment shown in FIG. 3, the stent graft 10 contacts the vascular tissue below the renal arteries 20, around the bifurcation 22 and at the iliac limbs 30 and extensions 28. In this embodiment, a seal is preferably achieved that will help exclude the entire aneurysmal region and, as a result, the hemodynamic pressures within the aneurysm 12 may be reduced.

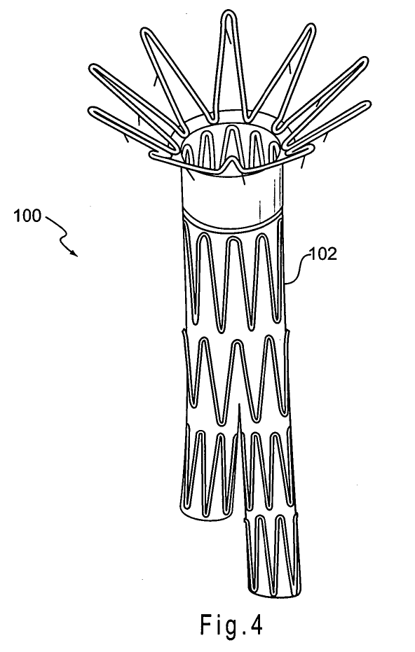

FIG. 4 shows another example of a modular bifurcated stent graft. This figure shows a three-piece modular bifurcated stent graft 100 also designed for deployment into an aorta.

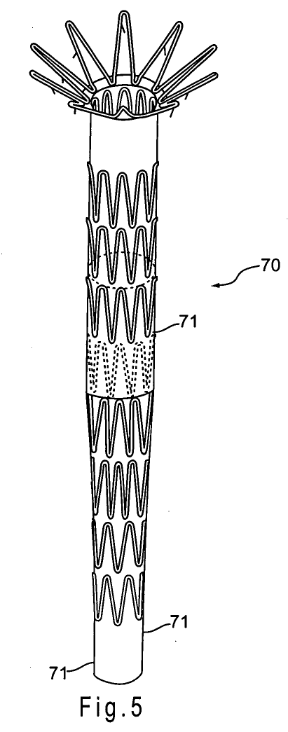

FIG. 5 shows a modular uni-iliac aortic stent graft 70 similar to that described in U.S. patent application Ser. No. 101,104,672, filed Mar. 22, 2002, which is incorporated herein by reference. A hydrophilic coating is posited on the graft fabric 71, reducing the diameter of the spaces between the fibers of the graft fabric.

Methods of Manufacture

A method for making an endoluminal graft prosthesis for implantation into a patient comprises the steps of providing a graft comprising a graft fabric having a plurality of yarns, the yarns having a denier of 5 to 50, and treating (coating or otherwise impregnating) the graft fabric with a hydrophilic material. In one embodiment, the graft fabric may be treated with bioactive agents in addition to the hydrophilic material. In another embodiment, the graft fabric may be treated with bioactive agents only. The method further comprises the step of attaching a stent to the graft fabric to form a stent graft. Means for attaching or affixing graft fabrics to stent(s) are well-known in the art. For example, the graft fabric may be sutured or glued to the stent(s). In one embodiment, the stent can be attached to the graft fabric prior to the step of treating the graft fabric with the hydrophilic material or bioactive agents. In another embodiment, the stent can be attached to the graft fabric following the step of treating the graft fabric with the hydrophilic material or bioactive agents. The method further includes the step of inserting the stent graft into an endovascular delivery system. The diameter of the delivery system is preferably from about 0.06 inches to about 0.27 inches, more preferably from about 0.10 inches to about 0.22 inches, and most preferably from about 0.13 inches to about 0.19 inches.

In one example, the hydrophilic material is applied to the graft fabric in any manner capable of coating the fabric. Hydrophilic material may be added to the porous graft fabric after preparation of the graft, for example by soaking, dipping, spraying, painting or otherwise applying the hydrophilic material to the graft. Dipping may be the preferred coating method. In this method, the graft fabric is dipped into a bath containing the hydrophilic material, causing the hydrophilic material to coat and, to some extent, impregnate the graft fabric. The coated graft fabric is then removed from the bath and allowed to dry. The thickness of the coating posited on the graft can be increased by repeating the dipping operation. Generally, the greater the amount of hydrophilic coated and/or impregnated, the lesser the porosity and the permeability of the graft fabric. The steps of dipping and drying are repeated until tests for permeability show the graft fabric to be sufficiently impervious to liquids. For certain clinical applications, such as the treatment of aneurysms, a permeability that is substantially equal to zero ml/min/cm2 is ideal, whereas for other applications, the permeability requirements may be less stringent, such as less than about 240 ml/min/cm2.

If it is desired to apply bioactive materials to the graft fabric, in addition to the hydrophilic material, this can be done before or after dipping, again depending on the desired clinical application.

Another method of applying hydrophilic coating may be by knife over roll techniques known to those skilled in the art.

Either before or after the graft fabric has been coated with the hydrophilic material and optional bioactive agents, standard methods can be used to affix the graft fabric to a supporting stent(s). As mentioned above, the graft material may be, for example, sutured or glued on the stent(s).

Delivery System

Delivery of a small diameter endoluminal graft prosthesis, such as a stent graft within a tortuous and small diameter vessel of a patient requires that the stent graft be packable into a suitably small delivery system that has sufficient pushability, trackability and lateral flexibility.

The prosthesis is delivered to the treatment site by endovascular insertion. Preferably, the endovascular delivery system is sufficiently rigid to enable the health practitioner performing the implantation procedure to push the delivery system deep into the vascular tree of a patient, but not so rigid as to cause vascular damage during the implantation procedure. Furthermore, preferably the delivery system would have enough lateral flexibility to allow tracking of the path of any one of the blood vessels leading to the implantation site.

A delivery system, or introducer, typically comprises a cannula or a catheter, having a variety of shapes according to the intended clinical application and implantation site. The graft or endoluminal prosthesis of this invention may be radially collapsed and inserted into the catheter or cannula using conventional methods.

In addition to the cannula or catheter, various other components may need to be provided in order to obtain a delivery system that is optimally suited for its intended purpose. These include and are not limited to various outer sheaths, pushers, stoppers, guidewires, sensors, etc. Examples of suitable delivery system were previously described and are known in the art. For example, an apparatus and methods of placing bifurcated stents have been described in U.S. Pat. No. 6,669,718. Also, U.S. Patent Application Publication Nos. 2005/0004663 and U.S. 20030149467A1, which are incorporated herein in their entirety, provide additional examples of available delivery systems. Another example of delivery system and method of delivering endoluminal devices, including extensions, was previously described in U.S. Pat. No. 6,695,875 B2, which is incorporated in its entirety.

In a preferred embodiment, an endovascular delivery system can deliver the graft, wherein the graft comprises a graft fabric comprising a plurality of yarns, the yarns having a denier of 5 to 50, 5 to 40, or 5 to 20. A preferred endovascular delivery system would have an outer diameter of about 0.13 inches to about 0.19 inches, more preferably an outer diameter of about 0.10 inches to about 0.22 inches, and most preferably an outer diameter of about 0.06 inches to about 0.27 inches (about 10 French to about 14 French).

In another embodiment, the delivery system can deliver the endoluminal prosthesis of the present invention, wherein the endoluminal prosthesis comprises the graft comprising a graft fabric. The graft fabric comprises a plurality of yarns, the yarns having a denier of from about 5 to about 50. More preferably, the yarns have a denier of from about 5 to about 40. Most preferably, the yarns have a denier of from about 20 to about 40. The endoluminal device may further comprise a stent supporting the graft fabric. The endoluminal device may comprise a plurality of stents. Further, to enable the endoluminal prosthesis to be packable in a small delivery system, for example, having diameter of about 0.06 inches to about 0.27 inches (about 10 French to about 14 French), it is preferable that at least most of the filaments or fibers making up the yarns of the graft fabric have a size of less than or equal to about 1.4 denier. More preferably, the size of the filaments or fibers less than or equal to about 0.7 denier, and most preferably, it is less than or equal to about 0.4 denier.

Preferably, such small yarns possess sufficient resistance to breakage in order to fulfill their role as an implant or transplant in the replacement or repair of blood vessel walls. The tenacity of a yarn, which is an expression of the ability of a yarn or fabric to resist breaking under tension, is preferably about 4 g per denier, or more preferably about 6 g per denier or more.

Once the prosthesis is deployed within a vessel, it expands and it can remain in place indefinitely, acting as a substitute vessel for the flow of blood or other fluids. Alternatively, if the prosthesis is intended for temporary treatment, it can be removed after a desired period of time from within the patient by conventional means.

In one embodiment, the invention is directed to a method for treating endovascular disease, such as aneurysm, and more specifically, an abdominal aortic aneurysm. The method comprises delivering an endoluminal implantable medical device comprising a stent; a tubular graft comprising a graft fabric supported by the stent. The graft fabric comprises a plurality of yarns, the yarns having a denier of from about 5 to about 50. More preferably, the yarns have a denier of from about 5 to about 40. Most preferably, the yarns have a denier of from about 20 to about 40.

### Alternative Embodiments

In one embodiment, the present invention is a graft material comprising a plurality of yarns, most of the yarns having a denier sufficient to form a graft packable in a delivery system having an outer diameter of from about 0.06 inches to about 0.27 inches, more preferably having an outer diameter from about 0.10 inches to about 0.22 inches, most preferably, having an outer diameter from about 0.13 inches to about 0.19 inches. The yarns have a denier of 5 to 50, 5 to 40, or 20 to 40. The filaments of the yarns have a denier less than or equal to about 1.4; less than or equal to about 0.7; or less than or equal to about 0.4. Preferably, at least one of the yarns comprises a monofilament. More preferably, at least one of the yarns comprises multifilaments. Preferably, at least one of the yarns is textured or non-textured. Preferably, the yarns comprise a synthetic polymer, such as a thermoplastic material. The thermoplastic material comprises at least one of polyester, polypropylene, polyurethane and polytetrafluoroethylene. Preferably, the yarns have a tenacity of about 4 grams per denier or more, about 6 grams per denier or more. The graft material may further comprise hydrophilic material posited on at least one surface of the graft. The hydrophilic material may be mechanically bonded to the surface of the graft. Alternatively, the hydrophilic material may be covalently bonded to the surface of the graft. Preferably, the yarns of the graft material are woven or nonwoven. The weave of the graft material may be a plain weave, a matt weave or a combination thereof. The weave type of the graft material may be uniform or non-uniform. The number of ends per inch is less than about 152 and the number of picks per inch is less than about 135.

In another embodiment, the invention is an endoluminal prosthesis, comprising a tubular graft material and a stent supporting the graft material. Preferably, the graft material comprises a plurality of yarns, each yarn having a denier sufficient to form a graft packable in a delivery system having an outer diameter of from about 0.06 inches to about 0.27 inches, preferably from about 0.10 inches to about 0.22 inches, and most preferably, from about 0.13 inches to about 0.19 inches. Preferably, the yarns of the graft have a denier of 5 to 50, 5 to 40, or 20 to 40. Preferably, the filaments of the yarns have a denier less than or equal to about 1.4; less than or equal to about 0.7; or less than or equal to about 0.4. Preferably, at least one of the yarns of the graft is a monofilament or multifilament. Preferably, at least one of the yarns is textured or non-textured. Preferably, the yarns of the tubular graft of the endoluminal prosthesis comprise a synthetic polymer. Preferably, the synthetic polymer is a thermoplastic material comprising at least one of polyester, polypropylene, polyurethane and polytetrafluoroethylene. Preferably, the yarns of the tubular graft of the endoluminal prosthesis have a tenacity of about 4 grams per denier or more, or about 6 grams per denier or more. The endoluminal prosthesis may further comprise a hydrophilic material posited on at least one surface of the graft. Preferably, the hydrophilic material may be mechanically bonded to the surface of the graft. Alternatively, the hydrophilic material may be covalently bonded to the surface of the graft of the endoluminal prosthesis. The yarns of the tubular graft of the endoluminal prosthesis may be woven or nonwoven. Preferably, the weave may be a plain weave, a matt weave, or a combination thereof. Preferably, the weave type of the yarns of the tubular graft of the endoluminal prosthesis is uniform or non-uniform. Preferably, the number of ends per inch is less than about 152 and the number of picks per inch is less than about 135. Preferably, the tubular graft material of the endoluminal prosthesis has, after implantation of the prosthesis into a vascular lumen of a patient, a permeability of about zero mL/min/cm2 to about 240 mL/min/cm2. More preferably, the tubular graft material of the endoluminal prosthesis has, after implantation of the prosthesis into a vascular lumen of a patient, a permeability of about 80 mL/min/cm2 to about 240 mL/min/cm2. Most preferably, the tubular graft material of the endoluminal prosthesis has, after implantation of the prosthesis into a vascular lumen of a patient, a permeability of about 160 mL/min/cm2 to about 240 mL/min/cm2.

In yet another embodiment, the present invention is a method for making a endoluminal graft prosthesis for implantation into a patient. The method comprises the steps of providing a graft material, providing a hydrophilic material, and coating the graft material with the hydrophilic material. The method further comprises the steps of supporting the coated graft material with a stent to form a stent graft, and inserting the stent graft into a delivery system. The diameter of the delivery system is preferably from about 0.06 inches to about 0.27 inches; more preferably from about 0.10 inches to about 0.22 inches; most preferably, from about 0.13 inches to about 0.19 inches. Preferably, the yarns of the graft material have a denier of 5 to 50, 5 to 40, or 20 to 40. Most preferably, the yarns have a denier less than or equal to about 1.4; less than or equal to about 0.7. Preferably, the filaments of the yarns have a denier less than or equal to about 0.4. Preferably, at least one of the yarns comprises a monofilament or multifilaments. Preferably, the yarns are textured or non textured. Preferably, the yarns have filaments comprising a synthetic polymer, such as a thermoplastic material. The thermoplastic material preferably comprises at least one material selected from the group consisting of polyester, polypropylene, polyurethane, and polytetrafluoroethylene. Preferably, the yarns have a tenacity of about 4 grams per denier or more; more preferably, about 6 grams per denier or more. Preferably, the step of coating the graft material with the hydrophilic material comprises positing the hydrophilic material on at least one surface of the graft material. Preferably, the step of coating the graft material with the hydrophilic material includes mechanically bonding the hydrophilic material to the surface of the graft. Alternatively, the step of coating the graft material with the hydrophilic material includes covalently bonding the hydrophilic material to the surface of the graft. Preferably, the yarns are woven or non woven. Preferably, the weave is one of a plain weave, a matt weave, or a combination thereof. Preferably, the weave type is uniform. Alternatively, the weave type may be non uniform. The number of ends per inch may be less than about 152 and the number of picks per inch is less than about 135.

It is to be understood that this invention is not limited to the particular methodology, protocols, animal species or genera, constructs, or reagents described and as such may vary. Other uses of the graft material of this invention will be apparent to those of ordinary skill in the art. It is also to be understood that the terminology used herein is for the purpose of describing particular embodiments only, and is not intended to limit the scope of the present invention which will be limited only by the appended claims. It is therefore intended that the foregoing detailed description be regarded as illustrative rather than limiting, and that it be understood that it is the following claims, including all equivalents, that are intended to define the spirit and scope of this invention.

1. A graft comprising a graft fabric comprising a plurality of yarns, the yarns having a denier of 5 to 50, said graft being packable in an endovascular delivery system having an outer diameter of from about 0.06 inches to about 0.27 inches.

2. The graft of claim 1, wherein the graft is packable in an endovascular delivery system having an outer diameter of from about 0.10 inches to about 0.22 inches.

3. The graft of claim 1, wherein the graft is packable in an endovascular delivery system having an outer diameter of from about 0.13 inches to about 0.19 inches.

4. The graft of claim 1, wherein the yarns are woven or non-woven.

5. The graft of claim 1, wherein the yarns have a denier of 20 to 40.

6. The graft of claim 1, wherein the yarns are made from filaments or fibers having a denier less than or equal to about 1.4.

7. The graft of claim 1, wherein the yarns are made from filaments or fibers having a denier less than or equal to about 0.7.

8. The graft of claim 1, wherein the yarns are made from filaments or fibers having a denier less than or equal to about 0.4.

9. The graft of claim 1, wherein the yarns comprise a synthetic polymer selected from the group consisting of polyester, polypropylene, polyurethane and polytetrafluoroethylene.

10. The graft of claim 1, wherein the graft fabric further comprises a hydrophilic material.

11. The graft of claim 10, wherein the hydrophilic material is mechanically bonded to a surface of the graft fabric.

12. The graft of claim 10, wherein the hydrophilic material is covalently bonded to a surface of the graft fabric.

13. The graft of claim 1, wherein the graft fabric further comprises a bioactive agent.

14. A graft comprising
a graft fabric comprising a plurality of yarns, the yarns having a denier of 5 to 50, the yarns being made from filaments or fibers having a denier less than or equal to about 1.4 and comprising a synthetic polymer; and
a hydrophilic material,
said graft being packable in an endovascular delivery system having an outer diameter of from about 0.06 inches to about 0.27 inches.

15. An endoluminal prosthesis, comprising
a tubular graft comprising a graft fabric comprising a plurality of yarns, the yarns having a denier of 5 to 50; and
a stent supporting the graft fabric,
wherein the endoluminal prosthesis is packable in an endovascular delivery system having an outer diameter of from about 0.06 inches to about 0.27 inches.

16. The endoluminal prosthesis of claim 15, wherein the yarns are woven or non-woven.

17. The endoluminal prosthesis of claim 15, wherein the yarns have a denier of 20 to 40.

18. The endoluminal prosthesis of claim 15, wherein the yarns are made from filaments or fibers having a denier less than or equal to about 1.4.

19. The endoluminal prosthesis of claim 15, wherein the graft fabric further comprises hydrophilic material.

20. An endoluminal prosthesis, comprising
a tubular graft, comprising a graft fabric comprising
a plurality of yarns, the yarns having a denier of 5 to 50, the yarns being made from filaments or fibers having a denier less than or equal to about 1.4, and comprising a synthetic polymer, and
a hydrophilic material; and
a stent supporting the graft fabric,
wherein the endoluminal prosthesis is packable in an endovascular delivery system having an outer diameter of from about 0.06 inches to about 0.27 inches.

21. A method of making an endoluminal graft prosthesis for implantation into a patient, comprising
providing a graft comprising a graft fabric having a plurality of yarns, the yarns having a denier of from 5 to 50;
treating the graft fabric with a hydrophilic material;
attaching a stent to the graft fabric to form a stent graft; and
inserting the stent graft into an endovascular delivery system having a diameter of from about 0.06 inches to about 0.27 inches.
